(19) United States
(12) Patent Application Publication
(10) Pub. No.: US 2012/0288454 A1
(43) Pub. Date: Nov. 15, 2012

(54) DENTAL PROTECTION TOOTHPASTE COMPOSITION

(76) Inventor: Wenzhong Zhao, Shanton (CN)

(21) Appl. No.: 13/519,582

(22) PCT Filed: Nov. 25, 2010
(86) PCT No.: PCT/CN10/79110

§ 371 Date: Jun. 27, 2012

(51) Int. Cl. A61K 8/97; A61Q 11/00; A61K 8/46; A61K 8/21; A61K 8/19

(52) U.S. Cl. 424/52; 424/58; 424/53; 424/57

(57) ABSTRACT

The present invention relates to chemical industry for daily use field, specifically a dental protection toothpaste composition. Except toothpaste matrix components, the toothpaste composition including (the following components by weight of total composition): 0.48%-3.3% six tissue salts for homeopathy, 0.1%-9.8% Chinese herb medicine extractant, 0.08%-1.3% at least one of vitamin C vitamin E and coenzyme Q10. The toothpaste composition ingredient of the present invention connects the homeopathy medicine such as cell tissue salts, Chinese herb medicine and vitamin etc. active ingredients, which will help for solving tooth and oral health problems.

### FIELD OF THE INVENTION

The present invention relates to chemical industry for daily use, particularly a dental protection toothpaste composition.

### BACKGROUND OF THE INVENTION

The application history of oral health products has already exceeded over one hundred years. The toothpaste is used most commonly which disperses the mild frictional powder into gel or soft paste, and adding cleanser to help cleaning the teeth, or adding fluoride to reduce teeth corrosion etc. In the real life, the supply of nutritive substances is usually through chewing and swallowing for the intestinal digestion and absorption. However, there is another kind of medical treatment method, which makes the certain medicine as the composite type to absorb directly through contacting of oral mucosa. Base on the principle and evidence of the certain medicine can be through oral mucosa, some nutritive substance or active ingredients are also proven to utilize this way for absorbing. Besides, the homeopathy in medicine aspect follows this principle to rehabilitate the patients at the beginning.

Although the oral health products emphasize the teeth cleaning, the supplement of fluoride or the dental protection function of Chinese herb medicine in varying degree, the medicine toothpaste is usually added Chinese herb medicine for treating mouth disease. The astragalus, sarcandrae glabra and panax pseudoginseng toothpaste etc., which are sold in the market have relief and therapeutical effect for the red and swollen gums, bad breath and dentine allergy etc. Identifiably, the certain nutritive substances or active ingredients have substantially effect for reducing of main prone mouth chronic disease and dental care.

Homeopathy as an alternative medicine is different with the Chinese and western medicine, it is an independent medical system. Chinese medicine studies the human body on overall angle and western medicine study on partially, but the homeopathy study on the microcosmic angle; which the principle is comply with the natural performance of the human body's physiology and pathology, and get the natural and peacefully to prevent and cure the disease through stimulating and adjusting the effect of human body self-healing system. In addition, it also characterizes in the high efficiency, fast, safety, non-toxic and side effects free. The father of medicine Hippocrates reminded the homeopathy as early as 400 BC and the homeopathy theory was excavated from ancient publications the by German Jews Dr. Samuel Hahnemann until 1790, then gradually improving so far as history of 100 years.

Dr. Wilhem Schüessler created the pioneering new homeopathy medical system. For the first, he applied all homeopathy medicine in the medical practice and found the tissue salts (cell salts) are existed in the blood and tissues of human body. They are a kind of inorganic biochemical elements and participating the various metabolic processes of bodies such as absorption and detox. Thus, the health of cells is inseparable with the participation of cell salts, the cells in the human body are under the good condition of nutrition can help building the health physical quality and have the effects of natural healing and recovery. Then, he expanded the homeopathy medicine to twelve kinds of cell salts step by step and developed a series of homeopathy based on the mineral tissue salts, including various kinds of different ratio of tissue salts and combination which used for the treatment of human diseases including dental health. Dr. Wilhem Schüessler definite the mineral tissue salts is alimentotherapy or nature therapy and considered the pathological symptoms will be generated when lacking the certain kind of tissue salts in the body. The best method to solve these symptoms is treat patients using highly diluted certain lacked tissue salts which can be fast absorbing. Therefore, the mineral salts are also used as the nutritional supplements for adjust the self-healing ability of body and get the curative effect of body health.

### SUMMARY OF THE INVENTION

The present invention designs the certain formula according to the principle of homeopathy which includes the ordinary used vitamins, a group of Chinese herb medicine components and the homeopathy tissue salts for keeping the teeth and oral health.

The present invention is achieved according to the following solutions: A dental protection toothpaste composition, excepting toothpaste matrix components, also includes (the following components by weight of total composition):

0.48%-3.3% six tissue salts for homeopathy;
0.1%-9.8% Chinese herb medicine extractant;
0.08%-1.3% at least one of vitamin C vitamin E and coenzyme Q10;
the homeopathy tissue salts are chosen from a six salts component including calcium fluoride, calcium phosphate, calcium sulfate, sodium phosphate, sodium chloride and silica, the combinative proportions of each of which is equal to each other according to their weight percentages. The six salts component as 1:3 weight percentages is dissolved into the solution whose weight percentage of water to ethanol is 4:1, to made for six salts component solution.

The Chinese herb medicine extractants includes 0.4%-4% propolis extracting solution, 0.2%-3.3% Chrysanthemum morifolium Ramat extracting solution, 0.05%-1.25% cranberry fruit extracting solution and 0.05%-1.25% liquorice extracting solution by weight of total composition.

The vitamin C in the present invention is chosen from calcium ascorbate powder and vitamin E is chosen from a-tocopheryl powder with wheat germ oil free.

The present invention also includes 0.0125%-2.5%-stevia rebaudiana extract, 0.05%-1.25% orange oil and 0.05%-1.25% lemon oil by weight of total composition.

The toothpaste composition in present invention, its matrix components includes: 10%-30% glycerol, 20%-40% calcium carbonate as abradant, 20%-40% silicon dioxide as excipient, and 0.5%-1.5% sodium lauroyl sarcosinate as vesicant by weight of total composition and the rest is deionized water.

The present invention according to the homeopathy as combat poison with poison principle, uses the activity integrant extracting from the plants, vitamins and coordinate the effects of homeopathy to simulate the self-healing ability of human body. The homeopathy in the present invention is not only for removing symptom or killing bacteria, but also for strengthens the self-healing ability of human body. The toothpaste composition formula in present invention combines the homeopathy medicine such as cell tissue salts, Chinese herb medicine and vitamins etc. activity integrants to help solving dental health problems.

In addition, some nutritive components in this formula can be directly absorbed by oral mucous, not through the alimentary system.

Finally, the composition formula includes toothpaste, oral paste and mouth wash etc., and the user can get the same health care effects similarly to take the tables.

### BRIEF DESCRIPTION OF THE DRAWINGS

The accompanying drawings facilitate an understanding of the various embodiments of this invention. In such drawings:

FIG. 1 is a tooth divided figure, wherein a is the lip face, b is the tongue face, c is mesial face and d is distal face; and

FIG. 2 is a record table of plague index number after teeth brushing used brand toothpaste in market and the toothpaste in present invention.

### DETAILED DESCRIPTION OF ILLUSTRATED EMBODIMENTS

The embodiment of the present invention is to provide a dental protection toothpaste composition including homeopathy mineral tissue salts and Chinese herb medicine etc. activity integrants, and can help to improve oral absorption. This toothpaste composition contains toothpaste matrix components, a group of homeopathy tissue salts within the certain biochemical effects for the patient's teeth and gum, a group of Chinese herb medicine extractant, at least one of vitamin E, vitamin C and coenzyme Q10 and the other optional components.

The present composition as toothpaste, excepting the teeth cleaning and oral fresh, it can also reinforce the immunoreactions of gum disease and tooth decay and promote the natural healing ability for the user. Although the present invention is preferred used in toothpaste composition, it also can be used together with mouth wash or chewing gum and coordinate with some carrier for processing the tooth, such as prevention cream or oral paste.

In an optional embodiment, the activity integrants includes matrix component, a group of homeopathy tissue salts, a group of Chinese herb medicine extractant, at least one of vitamin C, vitamin E and coenzyme Q10 and the other optional components.

The dental protection toothpaste in present invention, excepting matrix component, its main activity integrants includes a group of homeopathy tissue salt, a group of Chinese herb medicine, at least one of three vitamins and the other optional components, wherein the Chinese herb medicine comprises natural propolis, Chrysanthemum morifolium Ramat extracting solution, cranberry fruit extracting solution and liquorice extracting solution.

Propolis is an important product of honeybee, which as an aromatic and non-transparent gel solid mixture of resinous honeybees collect from tree buds or bark, their mandibular gland secretions and beeswax. It is used for building the sterile hive. Since it can prevent the bacterial infections, the propolis in the present invention as the ointment and used for healing abrade, bruise or mucosa after infection and promoting effect for immune system.

Another name of Chrysanthemum morifolium Ramat is chamomile. It is an extract of the traditional medical plant flower in China, commonly as light brown liquid which uses the water/glycerol or 10%-20% solution as the medium. Based on its good promotion for the immune system, the Chrysanthemum morifolium Ramat in the present invention can make the organism resisting the interference bacteria and virus. In addition, it also has the functions of inflammation reduction and lymphoid tissue excretion.

Cranberry fruit extracting solution includes large amount of Polyphenolic flavonoids which have strong antioxidant and antiviral effects. They are also used in preventing the bacteria in oral cavity and enhancing the oral aroma.

Glycerol is the dried root and rhizome of leguminous plants Glycy rrhiza uralensis Fisch., G. inf lata Bat. or G. glabra. L. It is a commonly used Chinese herb medicine in clinical, and its main active ingredients are glycyrrhizic acid and glycyrrhizic flavones. According to the record in Effective Botanical Ingredients Volumes [M]. (page 465, Beijing: People's medical press, 1986) at State Administration of Traditional Chinese herbal medicine Information Central station, the glycyrrhizic flavones is a kind of strong biological activity substance and has the usages at antiulcer, antibacterial, spasmolysis, hypolipidemic and analgesia etc. The glycerol extracting solution in the present invention is mainly used for assisting antiulcer in oral cavity and keeping dental and oral health.

As mentioned above, one or more kinds of physiological tissue salts can be used in the present invention coordinating with homeopathy.

There are twelve kinds of physiological tissue salts (also called cell salts). Since the German Dr. Schüessler invented these, it also called Schüessler salts. In an optimized formula, homeopathy salts includes several composition in twelve kinds physiological salts.

The roughly effects of twelve kinds of homeopathy tissue salts as following:

Calcium fluoride—Creating the elastic tissues and applying to over-relaxed elastic tissues, lack of teeth gloss, and reducing the activity of tooth decay.

Calcium phosphate—Balancing and keeping the most appropriate pH, helping maturation and mineralization of the tooth surface enamel and preventing the damage of tooth enamel.

Calcium sulfate—Purifying blood and oral tissues, and improving oral breath.

Iron phosphate—Relieving inflammation and promoting the oxygen transportation ability of blood.

Potassium chloride—Contributing to healing reaction activity of dental ulcer.

Potassium phosphate—Contributing to improve activity of gum tissue health.

Potassium sulfate—Promoting the cell metabolism and cell detoxification.

Magnesium phosphate—Being used for nourishing tissue.

Sodium chloride—Being used for improving the activity of salivary gland.

Sodium phosphate—As the acid acceptor and nutrition absorption auxiliary agent.

Sodium sulfate—Improving gum tissue and helping for cleaning the toxin in cells.

Silica—Excluding toxin in the tissue.

The dental protection toothpaste in present invention preferentially screens the homeopathy tissue salts which have healing and health care effects and further prefers to six salts as the following: Calcium fluoride, calcium phosphate, calcium sulphate, sodium phosphate, sodium chloride and silica.

The tissue salts in the embodiment of the present invention is the chosen six tissue salts component, wherein the combinative proportions of each salt are equal according to the weight percentages. The six salts component as 1:3 weight percentages dissolved into the water to ethanol solution 4:1 by weight percentage.

The formula in present invention, excepting the contained homeopathy tissue salts and Chinese herb medicine, and includes at least one of three vitamins as following: At first, vitamin C and coenzyme Q10 (ubiquinone). Vitamin C is existed as the sodium ascorbate or calcium ascorbate, and pH value appears neutral. It is an easily absorbed vitamin C after slightly grinding. Vitamin C in present invention is used for promoting the healing of oral gum diseases and reducing the accumulation of tea-stained teeth.

Furthermore, the vitamins of activity integrant can also contain vitamin E chosen from alpha tocopherol which is a dried powder without wheat germ oil∘Vitamin E in present invention is based on the known antioxidant characteristic and healing effect of gum tissue.

The components in present invention as an optional type, excepting the activity integrants, there is another optional component which includes one of the following: stevia rebaudiana extract, orange oil and lemon oil.

The stevia rebaudiana extract in present invention is stevioside which as a new natural sweetener extracting from the stevia of asteraceae herb. With the characteristics of high sweetness and low calorie, its sweetness as 200-400 times as sucrose and the caloric value is only one part per three hundred of sucrose, pure white color, proper taste, no off-smell, non-toxic side effects, no carcinogenic substance and food safety. Stevia rebaudiana extract in present invention is used for sweetener and tooth spot inhibitor, and balance the salty and bitter taste of vitamin C in toothpaste. Orange oil as a natural essential oil and odorant can be chosen for the optional components, lemon oil as well.

The toothpaste components in present invention, the matrix components of formula includes 5%-25% deionized water, 10%-30% Glycerol, 10%-30% Calcium carbonate and silicon dioxide as abradant, wherein 20%-40% calcium carbonate, 20%-40% silicon dioxide and 0.5%-1.5% sodium lauroyl sarcosinate as vesicant by weight of total composition.

The calcium component is comprised by edible calcium powder such as calcium carbonate and the silicon component is comprised by edible quartz after grinding which used for mild abradant and excipient.

Overall, the toothpaste formula in present invention contains 1.22%-22% activity integrant and 76%-98% matrix components. If there is necessary to add one or more kinds of optional components, its dosage range will be 0.1%-2% by weight of total toothpaste formula composition.

Therefore, a toothpaste composition which has activity integrants and optional components, the final usage amount of its activity integrants and optional components in sequence as following: 0.48%-3.3% homeopathy tissue salts (six salts type), 0.74%-9.8% a group of Chinese herb medicine extract which includes 0.4%-4% propolis extracting solution, 0.24%-3.3% Chrysanthemum morifolium Ramat extracting solution, 0.05%4.25% cranberry fruit extracting solution and 0.05%4.25% liquorice extracting solution; at least one of the vitamin activity integrands: 0.08%-1.3% Coenzyme Q10, 0.08%-1.3% vitamin C and 0.08%-1.3% vitamin E; the other optional components include: 0.0125%-2.5% stevia rebaudiana extract, 0.05%4.25% orange oil and 0.05%4.25% lemon oil by weight of total composition.

In present invention, the Chinese herb medicine extract mainly includes propolis extracting solution, Chrysanthemum morifolium Ramat extracting solution, cranberry fruit extracting solution and liquorice extracting solution.

The extracting method of propolis liquid as following: Since the propolis just collected from the hive out contains a lot of impurities such as wood flour and weeds and more invalid integrants like bee wax, the first process step is removing impurities. Then, freezing the propolis under 0° C. and smash to the powder, mixing the smashed prololis as ratio of 1 to 4 into the 95% ethanol solution. After soaking over 24 hours, filtering within filter cloth and removing impurities. Finally, the propolis ethanol extracting solution is obtained for later use.

The extracting method of Chrysanthemum morifolium Ramat extracting solution as following: After smash the Chrysanthemum morifolium Ramat, adding 15 times distilled water measured by weight and extracting twice which duration time is 30-40 minutes at 90° C. Merging the twice filtrate and putting into the rotary evaporator, reducing the pressure to 400-600 milli Hg, and the Chrysanthemum morifolium Ramat extracting solution will be obtained after concentrating to 0.25 g/ml at 55° C.

The extracting method of cranberry fruit extracting solution as following: using the 30% ethanol to clean the cranberry fruit and air drying, then adding the juiced cranberry fruit solution, 95% ethanol and 1,3-Butanediol as ratio of 1:1:1 and mixing intensively. After soaking over 24 hours, filtering within filter cloth and removing impurities. Finally, the cranberry fruit extracting solution is obtained for later use.

The extracting method of liquorice extracting solution as following: Extracting liquorice flavones through ordinary method and precisely weighting 1.00 g liquorice powder. Adding 70% ethanol and weighting, processing the soxhlet extraction twice and each duration time is 60 minutes. Then, weighting, complimenting the weight loss, filtering and merging collected solution. Measuring its integrated absorbance through UV-VIS spectrophotometer and calculating and reserving the content of liquorice flavones in liquorice extracting solution between 1.5%-1.8%.

The examples of the toothpaste formula as following, each component presents by weight percentage:

### Example 1

| | |
|---|---|
| Calcium carbonate | 32% |
| Silicon dioxide | 26% |
| Glycerol | 23% |
| Deionized water | 16.705% |
| Sodium lauroyl sarcosinate | 0.5% |
| Vitamin C | 0.08% |
| Vitamin E | 0.08% |
| Coenzyme Q10 | 0.08% |
| Propolis extracting solution | 0.4% |
| Chrysanthemum morifolium Ramat extracting solution | 0.24% |
| Cranberry fruit extracting solution | 0.05% |
| Liquorice extracting solution | 0.05% |
| Homeopathy tissue salts (six salts type) | 0.5% |
| Stevia rebaudiana extract | 0.015% |
| Orange oil | 0.17% |
| Lemon oil | 0.13% |

### Example 2

| | |
|---|---|
| Calcium carbonate | 32% |
| Silicon dioxide | 26% |
| Glycerol | 23% |
| Deionized water | 14.3% |
| Sodium lauroyl sarcosinate | 1% |
| Propolis extracting solution | 0.6% |
| Chrysanthemum morifolium Ramat extracting solution | 0.6% |
| Cranberry fruit extracting solution | 0.4% |
| Liquorice extracting solution | 0.4% |
| Homeopathy tissue salts (six salts type) | 1% |
| Vitamin C | 0.1% |
| Vitamin E | 0.2% |
| Coenzyme Q10 | 0.1% |
| Orange oil | 0.17% |
| Lemon oil | 0.13% |

### Example 3

| | |
|---|---|
| Calcium carbonate | 32% |
| Silicon dioxide | 26% |
| Glycerol | 23% |
| Deionized water | 13.1% |
| Sodium lauroyl sarcosinate | 1% |
| Propolis extracting solution | 0.6% |
| Chrysanthemum morifolium Ramat extracting solution | 0.7% |
| Cranberry fruit extracting solution | 0.3% |
| Liquorice extracting solution | 0.3% |
| Homeopathy tissue salts (six salts type) | 1.5% |
| Vitamin C | 0.2% |
| Coenzyme Q10 | 0.3% |
| Stevia rebaudiana extract | 1% |

### Example 4

| | |
|---|---|
| Calcium carbonate | 32% |
| Silicon dioxide | 26% |
| Glycerol | 23% |
| Deionized water | 12.3% |
| Sodium lauroyl sarcosinate | 1% |
| Vitamin C | 0.4% |
| Coenzyme Q10 | 0.2% |
| Homeopathy tissue salts (six salts type) | 1.8% |
| Propolis extracting solution | 0.5% |
| Chrysanthemum morifolium Ramat extracting solution | 0.8% |
| Cranberry fruit extracting solution | 1% |
| Liquorice extracting solution | 1% |

### Example 5

| | |
|---|---|
| Calcium carbonate | 32% |
| Silicon dioxide | 26% |
| Glycerol | 23% |
| Deionized water | 11.85% |
| Sodium lauroyl sarcosinate | 1% |
| Vitamin C | 0.2% |
| Vitamin E | 0.25% |
| Coenzyme Q10 | 0.15% |
| Propolis extracting solution | 0.5% |
| Chrysanthemum morifolium Ramat extracting solution | 0.25% |
| Cranberry fruit extracting solution | 0.75% |
| Liquorice extracting solution | 0.75% |
| Homeopathy tissue salts (six salts type) | 3.1% |
| Lemon oil | 0.2% |

### Example 6

| | |
|---|---|
| Calcium carbonate | 32% |
| Silicon dioxide | 26% |
| Glycerol | 23% |
| Deionized water | 13.4% |
| Sodium lauroyl sarcosinate | 1% |
| Vitamin C | 0.5% |
| Coenzyme Q10 | 0.2% |
| Propolis extracting solution | 0.6% |
| Chrysanthemum morifolium Ramat extracting solution | 0.8% |
| Cranberry fruit extracting solution | 0.5% |
| Liquorice extracting solution | 0.5% |
| Homeopathy tissue salts (six salts type) | 0.5% |
| Stevia rebaudiana extract | 0.7% |
| Orange oil | 0.17% |
| Lemon oil | 0.13% |

### Example 7

| | |
|---|---|
| Calcium carbonate | 30% |
| Silicon dioxide | 26% |
| Glycerol | 20% |
| Deionized water | 9.7% |
| Sodium lauroyl sarcosinate | 1% |
| Vitamin E | 1% |
| Coenzyme Q10 | 0.8% |
| Propolis extracting solution | 4% |
| Chrysanthemum morifolium Ramat extracting solution | 3.3% |
| Cranberry fruit extracting solution | 1.25% |
| Liquorice extracting solution | 1.25% |
| Homeopathy tissue salts (six salts type) | 1.5% |
| Orange oil | 0.2% |

### Example 8

| | |
|---|---|
| Calcium carbonate | 32% |
| Silicon dioxide | 26% |
| Glycerol | 22% |
| Deionized water | 11.25% |
| Sodium lauroyl sarcosinate | 1% |
| Coenzyme Q10 | 0.85% |
| Homeopathy tissue salts (six salts type) | 2% |
| Propolis extracting solution | 3.5% |
| Chrysanthemum morifolium Ramat extracting solution | 0.4% |
| Cranberry fruit extracting solution | 0.375% |
| Liquorice extracting solution | 0.375% |
| Stevia rebaudiana extract | 0.25% |

### Example 9

| | |
|---|---|
| Calcium carbonate | 33.5% |
| Silicon dioxide | 31% |
| Glycerol | 25% |
| Deionized water | 6.1% |
| Sodium lauroyl sarcosinate | 1% |
| Vitamin C | 0.8% |
| Homeopathy tissue salts (six salts type) | 0.5% |
| Propolis extracting solution | 0.5% |
| Chrysanthemum morifolium Ramat extracting solution | 0.4% |
| Cranberry fruit extracting solution | 0.5% |
| Liquorice extracting solution | 0.5% |
| Lemon oil | 0.2% |

### Example 10

| | |
|---|---|
| Calcium carbonate | 35% |
| Silicon dioxide | 30% |
| Glycerol | 25% |
| Deionized water | 6.4% |
| Sodium lauroyl sarcosinate | 0.5% |
| Vitamin E | 0.2% |
| Propolis extracting solution | 1% |
| Chrysanthemum morifolium Ramat extracting solution | 0.4% |
| Cranberry fruit extracting solution | 0.2% |
| Liquorice extracting solution | 0.2% |
| Homeopathy tissue salts (six salts type) | 0.5% |
| Stevia rebaudiana extract | 0.6% |

### Example 11

| | |
|---|---|
| Calcium carbonate | 35% |
| Silicon dioxide | 32% |
| Glycerol | 15.5% |
| Deionized water | 11% |
| Sodium lauroyl sarcosinate | 1% |
| Vitamin C | 0.2% |
| Propolis extracting solution | 1% |
| Chrysanthemum morifolium Ramat extracting solution | 0.4% |
| Cranberry fruit extracting solution | 0.9% |
| Liquorice extracting solution | 0.9% |
| Homeopathy tissue salts (six salts type) | 1.9% |
| Lemon oil | 0.2% |

### Example 12

| | |
|---|---|
| Calcium carbonate | 36% |
| Silicon dioxide | 31% |
| Glycerol | 15.5% |
| Deionized water | 8% |
| Sodium lauroyl sarcosinate | 1% |
| Coenzyme Q10 | 1.2% |
| Propolis extracting solution | 1% |
| Chrysanthemum morifolium Ramat extracting solution | 3% |
| Cranberry fruit extracting solution | 0.2% |
| Liquorice extracting solution | 0.2% |
| Homeopathy tissue salts (six salts type) | 1.9% |
| Stevia rebaudiana extract | 1% |

In addition, the formula in present invention also can be used in the oral paste ointment.

The following examples are the formula in present invention used in the oral paste ointment:

### Example 13

| | |
|---|---|
| Calcium carbonate | 29% |
| Silicon dioxide | 25% |
| Glycerol | 15% |
| Deionized water | 21.8% |
| Vitamin C | 0.55% |
| Vitamin E | 0.55% |
| Coenzyme Q10 | 0.55% |
| Propolis extracting solution | 2% |
| Chrysanthemum morifolium Ramat extracting solution | 2% |
| Homeopathy tissue salts (six salts type) | 1.55% |
| Cranberry fruit extracting solution | 0.7% |
| Liquorice extracting solution | 0.7% |
| Stevia rebaudiana extract | 0.3% |
| Orange oil | 0.17% |
| Lemon oil | 0.13% |

### Example 14

| | |
|---|---|
| Calcium carbonate | 29% |
| Silicon dioxide | 25% |
| Glycerol | 11% |
| Deionized water | 22.25% |
| Vitamin C | 1% |
| Vitamin E | 0.65% |
| Coenzyme Q10 | 1% |
| Propolis extracting solution | 3.8% |
| Chrysanthemum morifolium Ramat extracting solution | 1.4% |
| Cranberry fruit extracting solution | 0.7% |
| Liquorice extracting solution | 0.7% |
| Homeopathy tissue salts (six salts type) | 3% |
| Orange oil | 0.3% |
| Lemon oil | 0.2% |

### Example 15

| | |
|---|---|
| Calcium carbonate | 30% |
| Silicon dioxide | 28% |
| Glycerol | 12% |
| Deionized water | 22.45% |
| Vitamin C | 0.8% |
| Vitamin E | 0.65% |
| Coenzyme Q10 | 0.9% |
| Homeopathy tissue salts (six salts type) | 1% |
| Propolis extracting solution | 1% |
| Chrysanthemum morifolium Ramat extracting solution | 0.5% |
| Cranberry fruit extracting solution | 1.25% |
| Liquorice extracting solution | 1.25% |
| Lemon oil | 0.2% |

### Example 16

| | |
|---|---|
| Calcium carbonate | 29% |
| Silicon dioxide | 25% |
| Glycerol | 11% |
| Deionized water | 24.8% |
| Vitamin C | 0.8% |
| Coenzyme Q10 | 1% |
| Propolis extracting solution | 2% |
| Chrysanthemum morifolium Ramat extracting solution | 3.2% |
| Cranberry fruit extracting solution | 0.2% |
| Liquorice extracting solution | 0.2% |
| Homeopathy tissue salts (six salts type) | 1.8% |
| Stevia rebaudiana extract | 0.7% |
| Orange oil | 0.17% |
| Lemon oil | 0.13% |

### Example 17

| | |
|---|---|
| Calcium carbonate | 28% |
| Silicon dioxide | 25% |
| Glycerol | 10% |
| Deionized water | 24.9% |
| Vitamin E | 1.2% |
| Coenzyme Q10 | 1.2% |
| Propolis extracting solution | 4% |
| Chrysanthemum morifolium Ramat extracting solution | 3% |
| Homeopathy tissue salts (six salts type) | 0.5% |
| Cranberry fruit extracting solution | 1% |
| Liquorice extracting solution | 1% |
| Orange oil | 0.17% |

The production process of the present invention as following: Choosing the various needed compositions according to the different needed activity components and quantity of each.

1. Blending the needed vitamin C powder, vitamin E powder or coenzyme Q10 etc. activity components together, then grinding to the fine dry powder for later use.
2. Dissolving the six salt combinations into water/ethanol solution (the ration of 4:1) and getting the six salt combinations solution.
3. Mixing the needed glycerol, deionized water, homeopathy tissue salts solution, propolis extracting solution, Chrysanthemum morifolium Ramat extracting solution, cranberry fruit extracting solution, liquorice extracting solution, stevia rebaudiana extract, orange oil and lemon oil together and stirring fully.
4. Adding the fine dry powder in the step 1 slowly while instant stirring to the solution in step 3 until forming uniform slurry.
5. Adding the required amount of grinded calcium carbonate powder and silicon dioxide powder into the uniform slurry, stirring until all the powder fuse fully and forming uniform mushy or pasty composition which can be used in the toothpaste and oral paste (ointment) in present invention.

The toothpaste of present invention can reinforce the natural healing ability of oral tissue; effectively prevent periodontal diseases and good removing effect for the plague. The main component of plague is bacteria. When saliva mixing the food fragments and some epithelial cell peeled from oral intima are left on the tooth surface and gum juncture, it will breed a large amount of bacteria and form some soft yellowish-white scale which is plague. Plague is the reason of decayed tooth, gingivitis and periodontal diseases and very harmful to the dental health.

### Experimental Example

The effect contrast experiment of the toothpaste in present invention and brand toothpaste in market at the aspect of plague restrain:

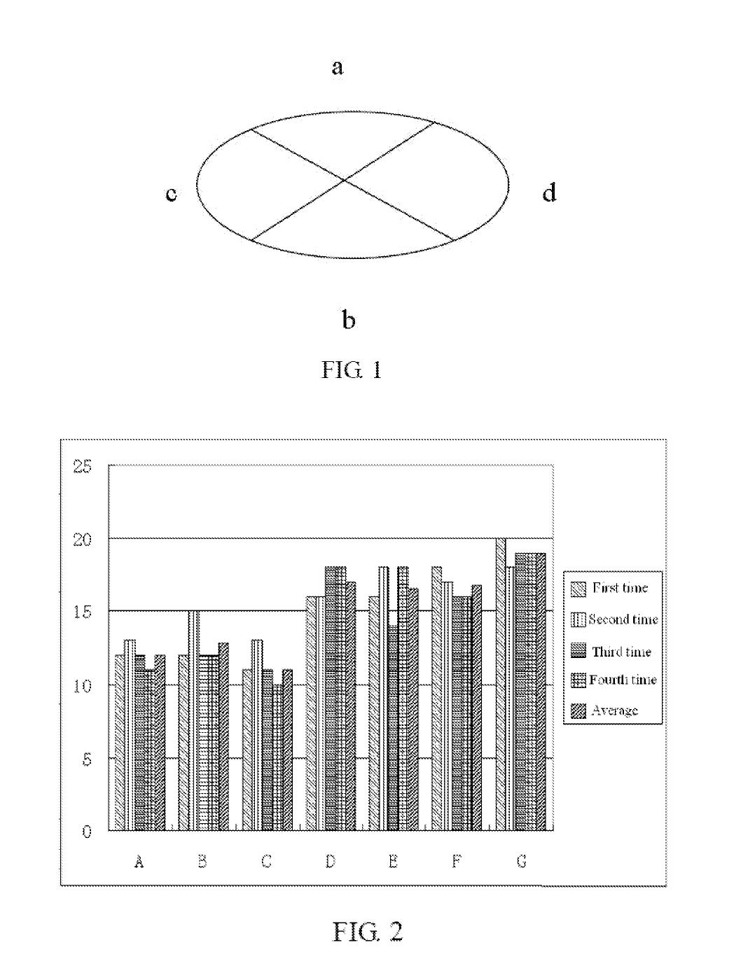

Establishment of the experimental method: As shown in FIG. 1, divide every tooth into four faces: a. lip face, b. tongue face, c. mesial face, d. distal face.

Experimental Method and Step Explanation:

1. Dipping cotton swab into plague indicator (choose 2% basic fuchsin as plague indicator) three droplet and daubing evenly on the teeth after brushing, rinsing the mouth with water after 30 seconds. Then, observing the colored spot on the teeth are plagues;

2. Observing the changed condition of the tooth color;

3. Dividing the tooth surface into four faces: mesial face, distal face, lip face and tongue face;

4. Calculating the percentage ratio of the plague face to all examined teeth, that is the plague index number;

5. The plague index number (mainly as distributed faces)=(the number of face which have plague)÷(examined tooth number×4 faces)×100%,

6. For example, there are 24 teeth for examination of Mr. Zhang and the faces after taking plague test is 16, so the plague index number of Mr. Zhang=16÷(24×4)×100%=16.7%;

7. Plaque index number within 20% is the normal range.

Compare the plague restraining effect of the toothpaste in present invention and brand toothpaste in market:

I Experiment Steps:
1. Getting the toothpaste of present invention (respectively sign as A, B, C) and brand toothpaste sale in market (respectively sign as D, E, F), and users brushing the teeth after dinner (about 20 minutes). (Note: The toothpaste sighed A, B, C are respectively are the composition formulas of Sample 1, Sample 2 and Sample 3, G is the matrix component in present invention which does not contain any activity integrant.)
2. Using the indicator to measure the index number of plague after teeth brushing used different toothpaste, and then recording these data.
3. Measuring four times on each kind of toothpaste, and calculating the average.

II Results and Discussion

Testing results are shown in Table 1 as below, and as FIG. 2 is the record chart and histogram of plague index number after teeth brushing used brand toothpaste in market and the toothpaste in present invention.

Table 1 is as below, showing the testing result.

| | Sample | | | | | | |
|---|---|---|---|---|---|---|---|
| | Sam- | Sam- | Sam- | Sam- | Sam- | Sam- | Sam- |
| | ple A | ple B | ple C | ple D | ple E | ple F | ple G |
| Time | Dental Plaque | | | | | | |
| First time | 12% | 12% | 11% | 16% | 16% | 18% | 20% |
| Second time | 13% | 15% | 13% | 16% | 18% | 17% | 18% |
| Third time | 12% | 12% | 11% | 18% | 14% | 16% | 19% |
| Fourth time | 11% | 12% | 10% | 18% | 18% | 16% | 19% |
| Average | 12% | 13% | 11% | 17% | 16.5% | 16.8% | 19% |

From the FIG. 2 histogram of plague index number after teeth brushing used brand toothpaste in market and the toothpaste in present invention: Although there are different plague restraining effect of the different brands and samples toothpaste, the examined plagues index number of sample A, B and C are all lower than the sample D, E and F of the brand toothpaste in market, and greatly lower than sample G (A matrix components toothpaste of present invention does not contain any activity integrands). It can be conclude that the dental protection toothpaste in present invention has better restraining effect to the bacteria, good removing function for plague and prevent the periodontal disease such as periodontitis, decayed tooth, and positive effect for the dental and oral health.

While the invention has been described in connection with what are presently considered to be the most practical and preferred embodiments, it is to be understood that the invention is not to be limited to the disclosed embodiments, but on the contrary, is intended to cover various modifications and equivalent arrangements included within the spirit and scope of the invention.

1. A dental protection toothpaste composition, comprising toothpaste matrix components, homeopathy six tissue salts for which accounts for 0.48% to 3.3% by weight, Chinese herb medicine extractant which accounts for 0.1%-9.8% by weight, and at least one of vitamin C vitamin E and coenzyme Q10 which account for 0.08%-1.3% by weight of the dental protection toothpaste composition;
wherein the homeopathy tissue salts are chosen from six salts component including calcium fluoride, calcium phosphate, calcium sulfate, sodium phosphate, sodium chloride and silica, the combinative proportions of each of which is equal to each other according to their weight percentages; the six salts component as 1:3 weight percentages is dissolved into the solution whose weight percentage of water to ethanol is 4:1, to made for six salts component solution;
wherein the Chinese herb medicine extractants comprises 0.4%-4% propolis extracting solution, 0.24%-3.3% Chrysanthemum morifolium Ramat extracting solution, 0.05%-1.25% cranberry fruit extracting solution and 0.05%-1.25% liquorice extracting solution by weight of said dental protection toothpaste composition.

2. The dental protection toothpaste composition according to claim 1, wherein the vitamin C is chosen from calcium ascorbate powder and the vitamin E is chosen from a-tocopheryl powder with wheat germ oil free.

3. The dental protection toothpaste composition according to claim 1, further comprises 0.0125%-2.5% stevia rebaudiana extract, 0.05%-1.25% orange oil and 0.05%-1.25% lemon oil by weight of said dental protection toothpaste composition.

4. The dental protection toothpaste composition according to claim 1, the toothpaste matrix components comprises 10%-30% glycerol, 20%-40% calcium carbonate as abradant, 20%-40% silicon dioxide as excipient, 0.5%-1.5% sodium lauroyl sarcosinate as vesicant by weight of said dental protection toothpaste composition and the rest is deionized water.

5. The dental protection toothpaste composition according to claim 2, further comprises 0.0125%-2.5% stevia rebaudiana extract, 0.05%-1.25% orange oil and 0.05%-1.25% lemon oil by weight of said dental protection toothpaste composition.
